(19) United States
(12) Patent Application Publication
(10) Pub. No.: US 2011/0151577 A1
(43) Pub. Date: Jun. 23, 2011

(54) DISPOSABLE DEVICE FOR AUTOMATED BIOLOGICAL SAMPLE PREPARATION

(76) Inventors: Jian-Ping Zhang, Moraga, CA (US); Samuel D. Chan, Daly city, CA (US); Daisuke Eto, Santa clara, CA (US); Takatoshi Hamada, Osaka (JP)

(21) Appl. No.: 13/060,433

(22) PCT Filed: Aug. 26, 2009
(86) PCT No.: PCT/US2009/004878

§ 371 Date: Feb. 23, 2011

Related U.S. Application Data
(60) Provisional application No. 61/092,013, filed on Aug. 26, 2008.

(51) Int. Cl. G01N 1/28; B67D 7/06; G01N 33/48

(52) U.S. Cl. 436/175; 222/81; 422/67

(57) ABSTRACT

A device and a process for preparing biological test samples are presented. The device has a disposable device with a connection mechanism for connecting to a closed sonication tube together with a filter capture unit to capture samples and to handle the samples without requiring additional tubes. With the device and/or method, samples are sonicated in the closed sonication tube that prevents aerosol contamination.

### DESCRIPTION OF THE INVENTION

1. Field of the Invention

A device, a system for the device, and a method for processing the device for preparing biological samples are presented. With the device and/or the method, samples are sonicated in a closed sonication tube that prevents air (airborne) contamination caused by sonicating a sample solution.

2. Background of the Invention

With biological sample preparation procedures, there has always been a concern regarding air (airborne) contamination from the sample. The present invention provides a hermetically sealable disposable tube device that addresses the issues of air (airborne) contamination while at the same time automates the process of purification of biological substances within the samples using the tube device.

### SUMMARY OF THE INVENTION

A device, a system for the device, and a method for processing the device for preparing biological test samples for assays are presented.

A solution processing disposable device includes a tube that is hermetically sealable for storing a sample solution therein and a unit that includes a base member for, at one end, receiving the tube and having a piercing member for piercing a (bottom and/or side) wall of the tube to allow for fluid communication between the tube and the base member; an absorbing member provided at an end of the base member opposite to where the tube is received, where the absorbing member allows absorption of substances in the sample solution; and a fluid communicating member attached to the base member at the end away from where the tube is received for allowing fluid to communicate with the base member through the absorbing member. The term “absorption” is used here in general to include “adsorptions” and other types of sorption processes.

An automatic sample solution treatment system includes a plurality of tubes each with a slidable plunger to hermitically seal a sample inside; a robotic arm for transporting the tubes; a sonicator horn for accepting the tubes to sonicate the tubes; a plurality of units respectively having piercing members, where each piercing member is in a space for receiving the tube, respectively having fluid communicating members, where each fluid communicating member is at an end opposite where the tube is to be received, and respectively having absorbing members for binding substances in the sample solution; a gripping mechanism attached to the robotic arm for gripping the tubes so that the tubes can be securely pressed by the robotic arm into the respective units to cause the piercing members of the respective units to pierce the bottom and/or side wall of the tubes and to thereby combine the tubes and the respective units to form single disposable tube devices, wherein for each disposable tube device, fluid communication between the tube and the fluid communicating member of the unit is made through the absorbing member; vials containing buffers and reagents for the robotic arm to position the disposable tube devices over the vials; and a plunger actuator for moving the plunger of each disposable tube device to draw in the buffer or reagents from the fluid communicating member and through the absorbing member or to expel fluid such as the sample solutions, buffers and reagents inside each device through the absorbing member and out through the fluid communicating member.

A method of treating sample solution includes providing a plurality of sample solutions for assays into respective tubes; sealing hermetically each tube with an air control mechanism; applying the tubes to a sonicator; transporting the tubes out of the sonicator to engage with respective units each having a space for receiving the tube and having, opposite the side where the tube is received, a fluid communicating member for drawing or expelling fluid such as the sample solutions, buffers and reagents, wherein each unit has a piercing member at the space for receiving the tube; engaging the tubes respectively to the spaces of the units to form single disposable tube devices; piercing the tubes respectively by the piercing members of the units to allow fluid communication between the tubes and the respective units; positioning the disposable tube devices over to a rack with wells and/or vials of buffers and reagents; actuating the air control mechanism of the devices relative to the devices to draw in fluid or expel fluid; absorbing the substances in the sample solutions to the absorbing members of the devices by moving the plungers to force the samples through the absorbing members; eluting the absorbed substances from the absorbing members by drawing in elution buffer from the wells and/or vials; and positioning the devices over the wells and/or vials with reagents and expelling the substances in the elution buffer to the wells and/or vials.

Another method of treating sample solutions including providing a plurality of sample solutions for assays into respective tubes; sealing hermetically each tube with an air control mechanism; transporting the tubes to engage with respective units each having a space for receiving the tube and having, opposite the side where the tube is received, a fluid communicating member for drawing in or expelling fluid such as the sample solutions, buffers and reagents, wherein each unit has a piercing member at the space for receiving the tube; engaging the tubes respectively to the spaces of the units to form single disposable tube devices; applying the disposable tube devices to a sonicator; piercing the tubes respectively by the piercing members of the units to allow fluid communication between the tubes and the respective units; positioning the disposable tube devices over to a rack with wells and/or vials of buffers and reagents; actuating the air control mechanism of the devices relative to the devices to draw in fluid or expel fluid; absorbing the substances in the sample solutions to the absorbing members of the devices by moving the plungers to force the samples through the absorbing members; eluting the absorbed substances from the absorbing members by drawing in elution buffer from the wells and/or vials; and positioning the devices over the wells and/or vials with reagents and expelling the substances in the elution buffer to the wells and/or vials.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the invention and together with the description, serve to explain the principles of the invention.

### BRIEF DESCRIPTION OF THE DRAWINGS

FIG. 1 shows an embodiment of a disposable tube device according to the present invention.

FIGS. 2A-2M shows an embodiment of a method of processing a disposable tube device according to the present invention.

FIG. 3 shows an embodiment of a system for processing a disposable tube device according to the present invention.

FIG. 4 shows an embodiment of an array of buffers and reagents according to the present invention.

FIG. 5A to 5C show another embodiment of a disposable tube device according to the present invention.

FIGS. 6A to 6D show another embodiment of a disposable tube device according to the present invention.

FIGS. 7A to 7E show an embodiment of a robotic arm according to the present invention.

FIG. 8 shows a schematic diagram of a system for processing one or more disposable tube devices according to the present invention.

FIG. 9 shows an example of a mechanical base that provides a robotic arm movements.

FIGS. 10A and 10B shows respectively an example of a protocol and an exemplary diagram of reagent location in a rack according to the present invention.

FIGS. 11A and 11B further shows another embodiment of the present invention.

FIG. 12 shows an exemplary display of the present invention.

FIGS. 13A and 13B show the results of an example of the present invention.

### DESCRIPTION OF THE EMBODIMENTS

Reference will now be made in detail to the present exemplary embodiments of the invention, examples of which are illustrated in the accompanying drawings. Wherever possible, the same reference numbers will be used throughout the drawings to refer to the same or like parts.

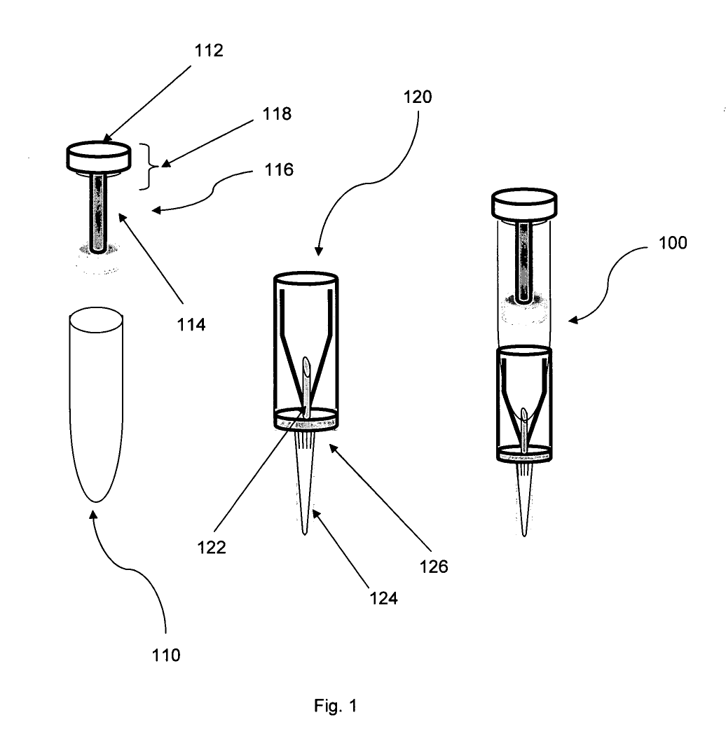

An embodiment of a disposable tube device is described below. The disposable tube device may be composed of one or more components but an example of a two component system will be described below. A disposable tube device 100 as shown in FIG. 1 is in two parts: a sonication tube 110 and a filter unit 120.

As shown in FIG. 1, the sonication tube 110 is configured in a way for it to be inserted securely into a sonicator (not shown) for the sample to be sonicated. At one end of the sonication tube 110, the sonicator tube is designed to accept a plunger 112 along with a plunger spacer 114 to act as a stopper or a spacer for the plunger during sonication. The plunger 112 having a cap 116 is pre-assembled to form a single cap unit 118. The sonication tube 110 is capped with the cap unit 118. The cap unit 118 forms an airtight closure with the tube 110. The cap unit 118 is a single use disposable part together with the tube 110. The other end of the sonication tube 110 can be pierced or punctured with a sharp object such as a needle or a pointed rod such as a pointed plastic rod (hollow needle).

Also shown in FIG. 1, the filter unit 120 is a tube where one end is open to receive the sonication tube 110. Inside the unit 120, there is a piercing member 122, which may be a needle or a pointed plastic rod or other sharp objects that will pierce the sonication tube 110 at the end opposite to where the plunger is situated once the sonication tube is fully inserted into the unit. At the other end of the unit 120, there is a tapering tube 124 for communicating with the sonication tube 110 through the piercing member 122 as a fluid communicating member. Between the tapering tube 124 and the piercing member 126, there is a membrane 126 made of, for example, a nucleic acid (NA) binding material (e.g. a DNA or RNA binding material). Therefore, the disposable tube device 100 is configured so that liquid will pass through the membrane 126 when liquid is transported between the sonication tube 110 and the tapering tube 124. Liquid such as sample solutions, reagents or buffer can be drawn in or expelled through the tapering tube 124. The sample solution contains substances that may be DNA, RNA, proteins, or other molecules targeted for purification. All of the components may be made of plastic with the possible exception of the piercing member 122, which can be made of metal or plastic, and the membrane 126 for binding substances such as nucleic acids, or a bound substance thereof. The membrane 126 can be any filters such as an ultra thin membrane for capturing nucleic acids or other solid media used for nucleic acid purification. For other types of substances different absorbing materials may be appropriately selected, for example, a column matrix or resin may be used to bind the target substances in the solution. Particles with an iron core and encapsulated by silicone may be provided in the solution to adhere target substances on the particles and then the target substances adhered to the particles may be magnetically separated. Also, a plain glass fiber membrane can be used for filtering out large loads of DNA binding particles or can be used to directly bind DNA.

In the case the membrane 126 is made of the NA binding material, the buffers and reagents for the above disposable tube device may be chosen appropriately for that purpose.

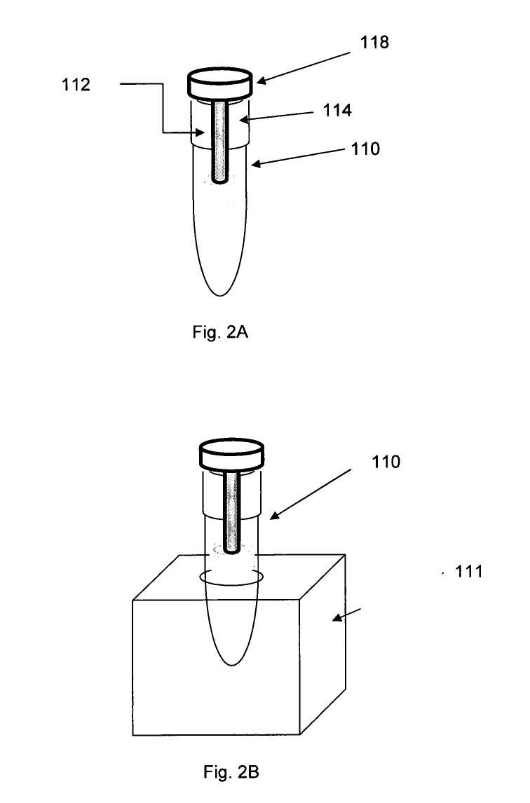

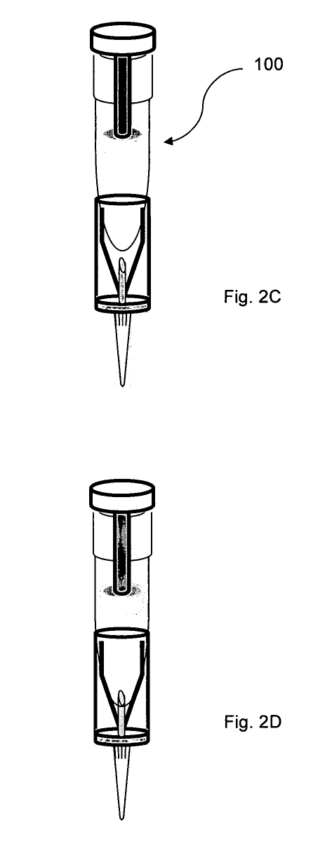

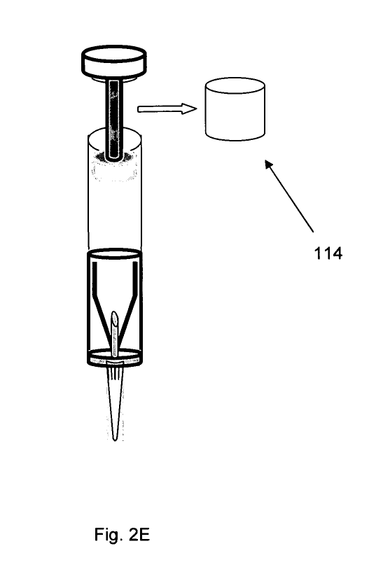

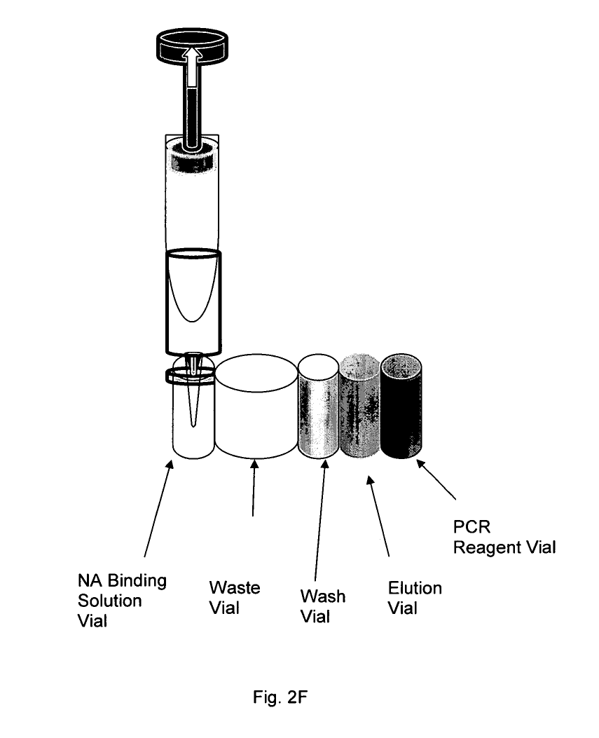

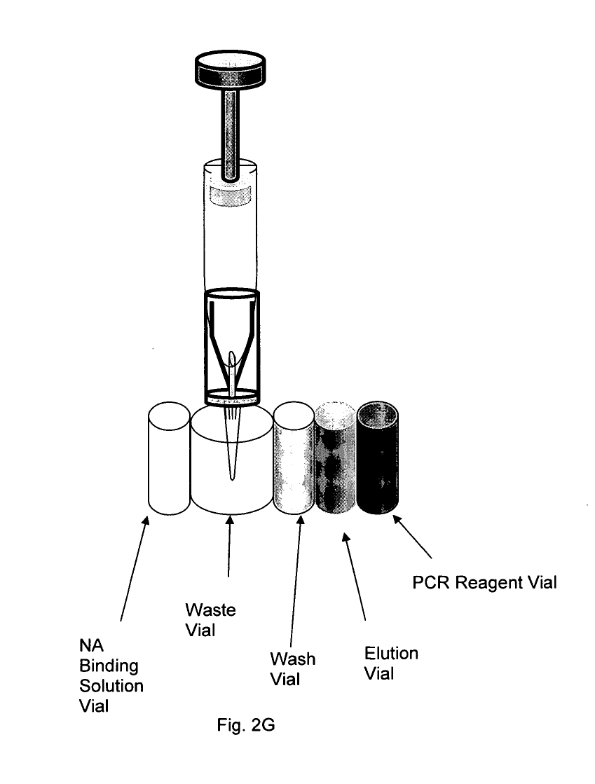

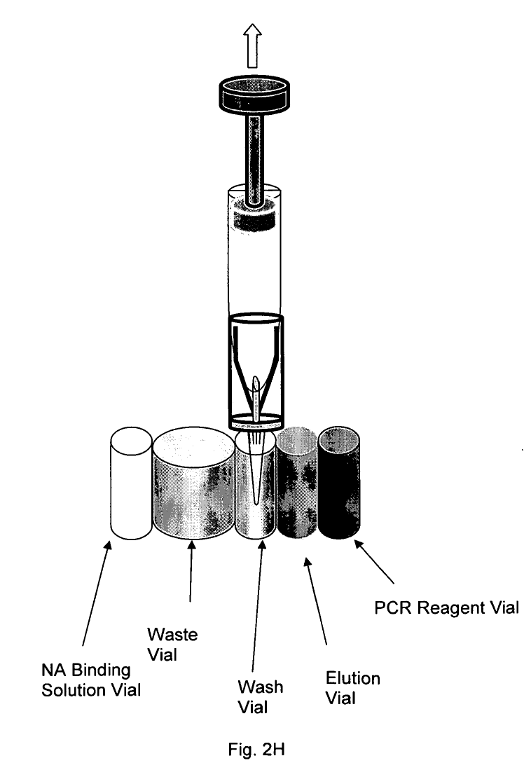

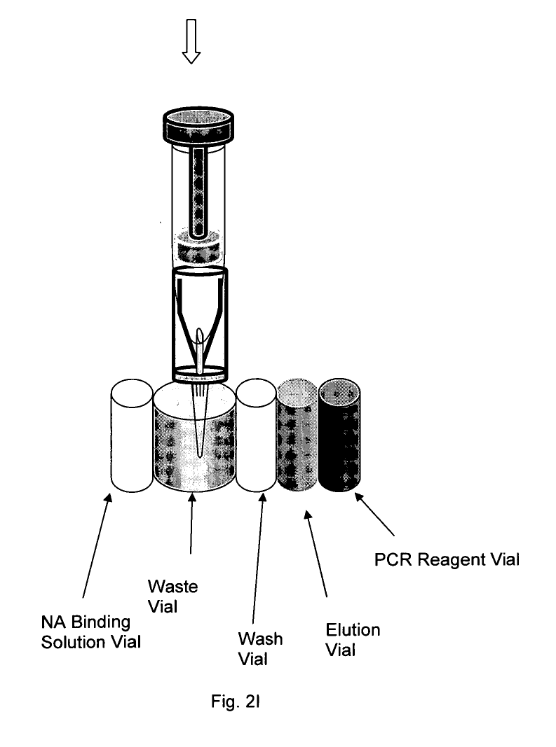

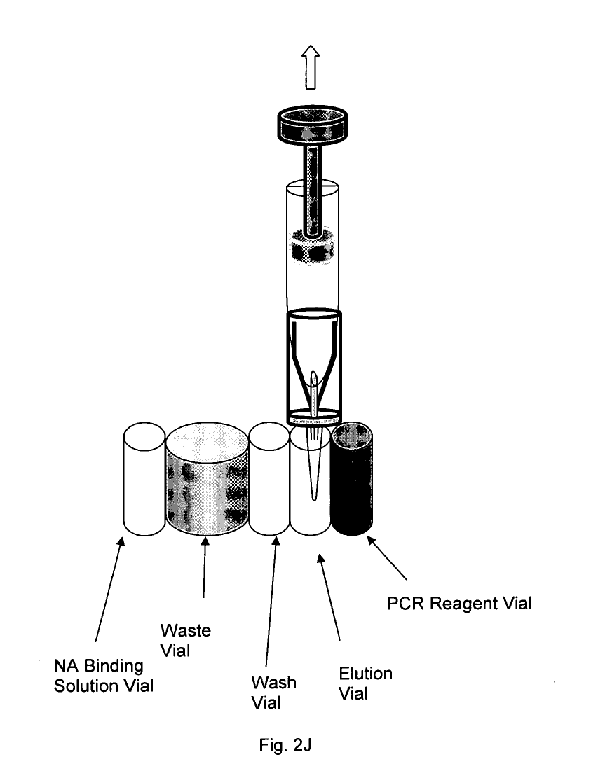

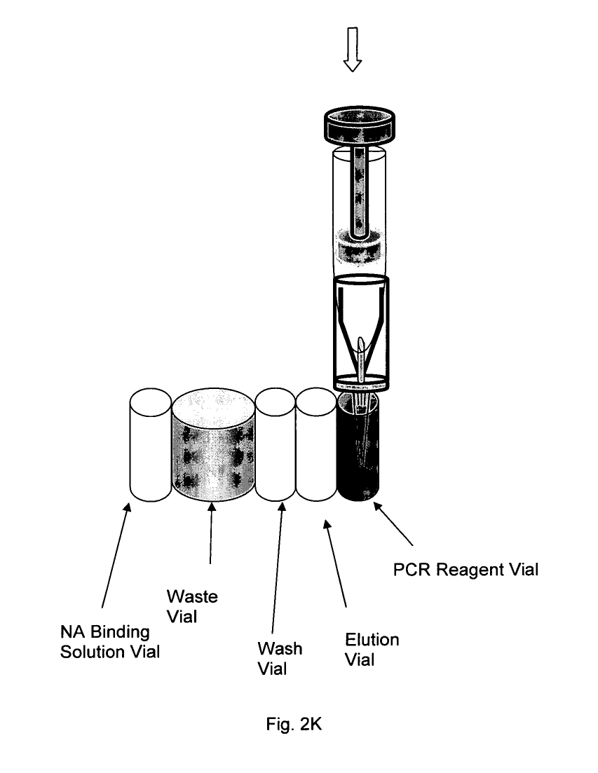

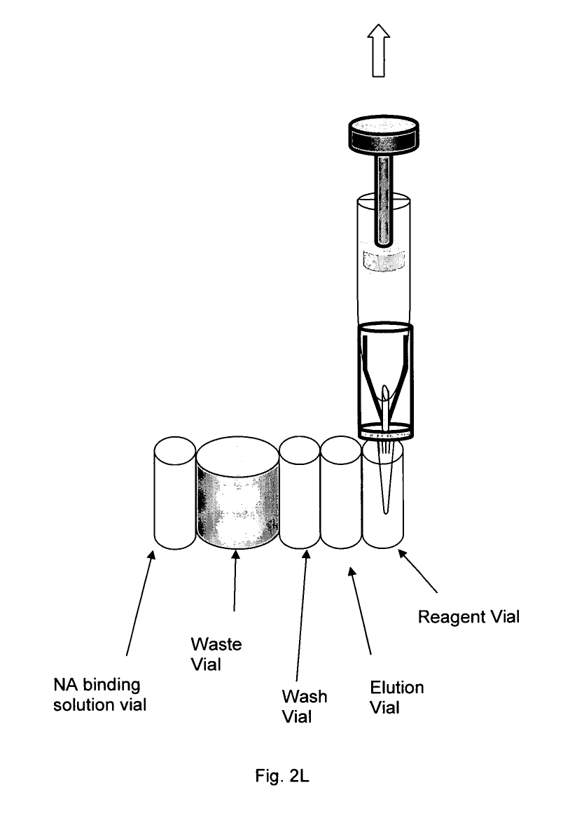

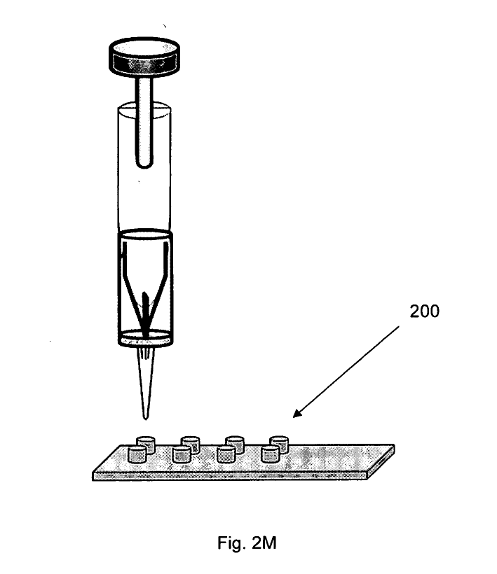

The following describes an embodiment of how the disposable tube device 100 can be engaged to prepare a test sample such as purified nucleic acid. Although the description below is explained in terms of one disposable tube device 100, there can be more than one device being processed at the same time. The following procedure is explained in reference to FIGS. 2A-2L.

The volume of the input sample solution in the sonication tube 110 can be, for example, from 100 ul to 1 ml. The output of, for example, purified nucleic acid volume extracted from the sample solution by using an elution buffer can be about 25 to 50 ul (with, for example, PCR reagents). The total prep time can be less than 20 minutes.

The sonication tube 110 is used to host a sample solution. The sample solution may be pretreated before being provided into the sonication tube 110. The sonication tube 110 with the sample solution is loaded onto a sample rack. The volume of the input sample solution has to be limited so that the sample will not overload the NA binding capacity of the membrane 126.

After the sample solution has been inserted into the sonication tube 110, the plunger 112 with the cap unit 118 and the spacer 114 are provided to the tube to form an airtight seal (FIG. 2A).

The sonication tube 110 is then applied to a sonicator 111 for lysing cells (e.g E. coli cells) within the sample solution so that nucleic acid material from the lysed cells will be released into solution (FIG. 2B). Glass beads may be provided within the sample solution to provide mechanical forces coupled with sonication forces to facilitate cell lysis.

The sonication tube 110 is then transported out of the sonicator. The sonication tube 110 is now ready to be connected with the filter unit 120.

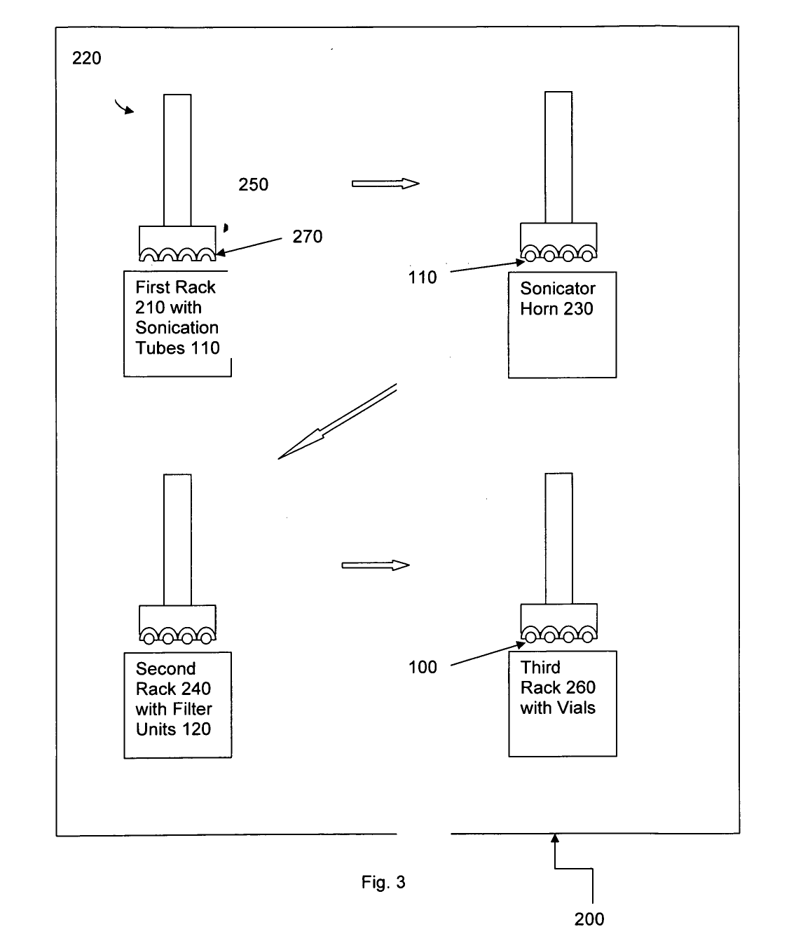

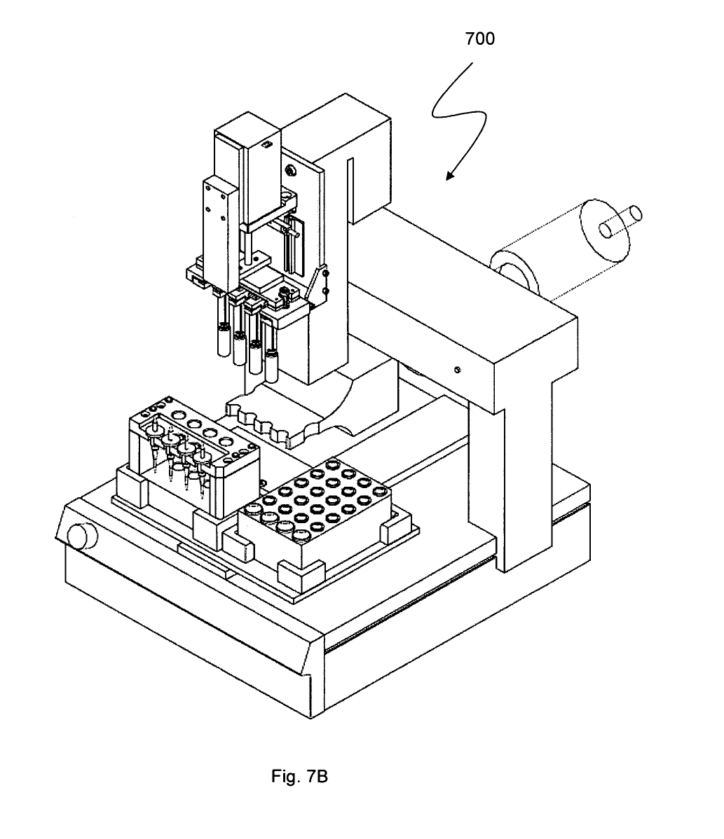

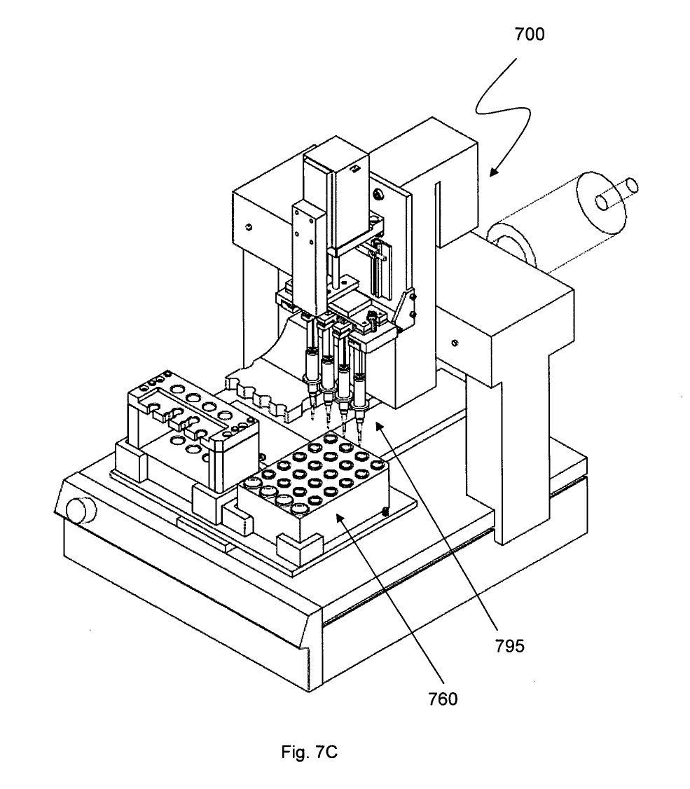

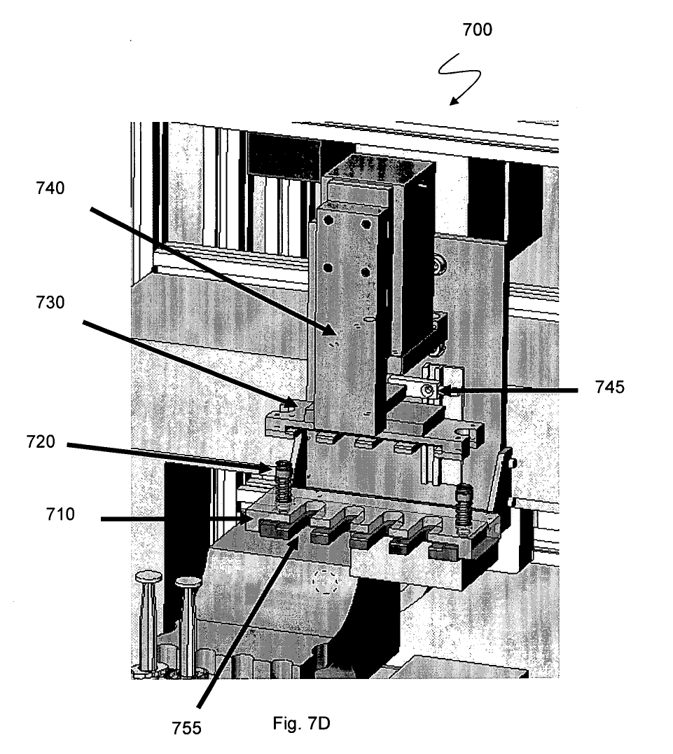

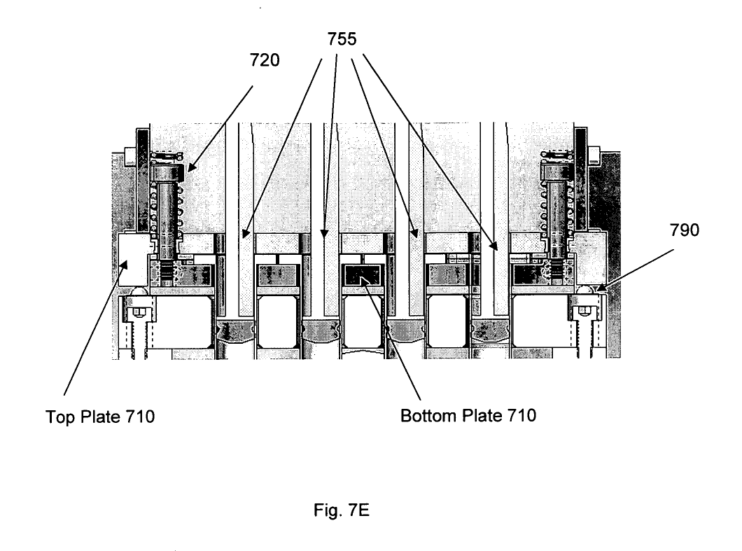

The sonication tube 110 is positioned over the filter unit 120 and then pushed to lock into the filter unit (FIG. 2C). Furthermore, by further forcing the tube 110 into the filter unit 120, the end of the tube 110 opposing the cap unit 118 is pierced by the piercing member 122 to allow communication between the sonication tube 110 and the filter unit 120 (FIG. 2D). The plunger spacer 114 is removed so that the plunger 112 can be moved up or down within the sonication tube 110 (FIG. 2E). The plunger spacer 114 may also be removed earlier after placing the sonication tube 110 in the sonicator in an automated operation. A negative pressure may be applied within the sonication tube 110 by pulling on the plunger 112 by a plunger actuator 270 (see FIG. 3 for a partial view; for a 3D rendition of the actuator see 740 in FIGS. 7A to 7E) to prevent any liquid leakage while the tube is being pierced. The sonication tube 110 and the filter unit 120 together form a single unit, which is the disposable tube device 100.

The disposable tube device 100 is placed over a nucleic acid binding solution vial and/or wells. By pulling back the plunger, the device draws in the binding solution, and mixes it with the solution containing lysed cells (the sonicated sample solution) (FIG. 2F). Further mixing may be performed by expelling the fluid back to the nucleic acid binding solution vial and/or wells and drawing the fluid back into the device one or more times. FIG. 2F further shows a sample preparation reagent cartridge 130, which is a combination of, for example, a binding buffer vial, a waste vial, a wash vial, an elution vial, and a PCR, reagent vial. Other types of buffers and reagents may be used depending on the target substances to be separated. The cartridge is preloaded with measured sample preparation reagents for a single sample preparation operation. The vials can be sealed with a membrane on top that prevents liquid evaporation during storage.

Then the device 100 is repositioned over a waste vial and the plunger 112 is pushed down to force the liquid through the membrane 126 into a waste vial (FIG. 2G). The nucleic acid in the cell lysis solution is captured by the membrane 126 designed for NA binding.

The filter unit 120 functions to eliminate any potential air contamination by aerosol generated during sonication. Its filtration function can also capture any surviving pathogenic bacterial after the sonication process.

The disposable tube device 100 is positioned over a wash vial to draw in the wash buffer (FIG. 2H), and then positioned over the waste vial to expel the washing solution from the device (FIG. 2I). This process may be repeated one or more times.

The disposable tube device 100 is then positioned over an elution buffer vial to draw in the elution buffer (FIG. 2J), and then positioned over a PCR reagents vial to elute the purified nucleic acid solution into the PCR reagents (FIG. 2K) and then the nucleic acid/PCR reagent mixture is drawn up (FIG. 2L). The nucleic acid/PCR reagent mixture is now ready to be delivered to, for example, a well in a PCR chip 200 for PCR assay (FIG. 2M). Although the example here referred to preparations of nucleic acids, the device is not limited to this. If the isolated substances are not nucleic acids, the other types of assay that are suitable for the analysis of the isolated substances may follow.

The above example described a system where one disposable tube device was processed, but below, a system that processes simultaneously more than one disposable tube device 100 is described.

In FIG. 3, there is shown an exemplary system 200 including a first rack 210 for accepting a plurality of sonication tubes 110, a robotic arm 220 for transporting the sonication tubes 110 all together, a sonicator horn 230 for accepting the sonication tubes 110 to sonicate the tubes, a second rack 240 for containing a matching plurality of filter units 120, a gripping mechanism 250 attached to the robotic arm 220 for gripping the sonication tubes 110 so that the tubes can be securely pressed by the robotic arm 220 into the matching filter units 120 provided in the second rack 240 to cause the piercing members 122 (see FIG. 1) to pierce the bottom or side wall of the sonication tubes 110 and to combine the sonication tubes 110 and the filter units 120 to form single disposable tube devices 100, a third rack 260 for holding various vials and/or providing wells containing buffers and reagents so that the robotic arm 220 can reposition the disposable tube devices 100 over the appropriate vials and/or wells, and a plunger actuator 270 to draw in the buffer or reagents or to expel the liquid inside the devices 100 through tapering tubes 124 (see FIG. 1) of the disposable tube devices 100.

The following is one embodiment of how the system 200 operates to prepare samples for assays. FIG. 3 shows four disposable tube devices 100 being processed simultaneously but this number is merely exemplary and not limited to four. The system can be designed to process a plurality of disposable tube devices. Each of the disposable tube devices in FIG. 3 is processed in the same way as described in FIG. 2A-K so any elements not shown in FIG. 3 are shown in FIGS. 2A-K.

The first rack 210 is designed to accept sample solutions, such as a solution containing cells of E. coli, provided in the sonication tubes 110. The sample solutions may be pretreated before being provided into the sonication tube 110. After the sample solution has been inserted into the sonication tube 110, cap units 118 are provided to the tubes respectively to form an airtight seal. The sonication tubes 110 containing the sample solutions are loaded onto the first rack 210.

The robotic arm 220 then transports the sonication tubes 110 from the first rack 210, then transports the sonication tubes 110 to the sonicator horn 230, and deposits them therein to lyse cells in the sample solution by sonication so that nucleic acid material from the lysed cells will be released into the sample solution. Glass beads may be provided within the sample solution to provide mechanical forces coupled with sonication forces to facilitate cell lysis. The sonicator horn 230 is a dry block that has wells to securely accept the sonication tubes 110 and does not use liquid to transfer the sonication forces. No sonication probe is necessary to directly contact the samples within the tubes 110 during operation. A sound insulator may be provided to reduce operation noise.

The robotic arm 220 then grips the sonication tubes 110 by the griping mechanism 250 and transports the sonication tubes 110 out of the sonicator horn 230. The sonication tubes 110 are now ready to be connected with the filter unit 120.

The robotic arm 220 positions the sonication tubes 110 over the respective filter units 120 provided in the second rack 240 and the pushes the sonications tubes 110 to lock into the respective filter units. The end of the tubes 110 opposite where cap units 118 are positioned are pierced by piercing members 122 in the filter units 120 to allow communication between each set of the sonication tube 110 and the filter unit 120. The plunger spacers 114 are removed so that the plungers 112 can be moved up or down within the sonication tube 110 by the plunger actuator 270. A negative pressure may be applied within the sonication tubes 110 by pulling on the plunger 112 by the actuator 270 to prevent any liquid leakage while the tubes are being pierced. The sonication tube 110 and the filter unit 120 together form a single unit, which is the disposable tube device 100.

The robotic arm 220 then transports the disposable tube devices 100 over respective nucleic acid binding solution vials and/or wells provided in the third rack 260. The plunger actuator 270 pulls back the plungers 112, which draws in the binding solution and mixes it with the sonicated sample solution containing lysed cells. Further mixing may be performed by expelling the fluid back to the nucleic acid binding solution vial and/or wells and drawing in the fluid back into the device one or more times. Then the robotic arm 220 repositions the disposable tube devices 100 over respective waste vials and/or wells provided in the third rack 260, and the plunger actuator 270 pushes the plungers 112 down to force the liquid through respective membranes 126 in the filter units 120 into the waste vials and/or wells. The nucleic acid in the cell lysis solution (sonicated sample solution) is captured by the membranes 126 designed for NA binding.

The filter units 120 function to eliminate any potential air (or airborne contamination by aerosol generated during sonication as the disposable tube device is hermetically sealed. Its filtration function can also capture any surviving pathogenic bacterial after the sonication process.

The robotic arm 220 then positions the disposable tube devices 100 over respective washing buffer vials and/or wells also provided in the third rack 260 to pick up washing buffer and then positions the devices 100 over the waste vials and/or wells to remove the washing solution from the devices 100. This process may be repeated one or more times.

The robotic arm 220 then positions the disposable tube devices 100 over respective elution buffer vials and/or wells in the third rack 260 to draw in the elution buffer, and then the robotic arm 220 positions the devices 100 over respective PCR reagents vials and/or wells in the third rack 260 to elute the purified nucleic acid solution into the PCR reagents. Following this, the reagent is drawn in to produce a nucleic acid/PCR reagent mixture. The nucleic acid/PCR reagent mixture is now ready to be delivered into a PCR chip for PCR assay.

The volume of the input sample solution in each sonication tube 110 can be, for example, from 100 ul to 1 ml. The output purified nucleic acid volume extracted from each sample solution can be about 25 to 50 ul (with, e.g., PCR reagents). The total prep time can be less than 20 minutes.

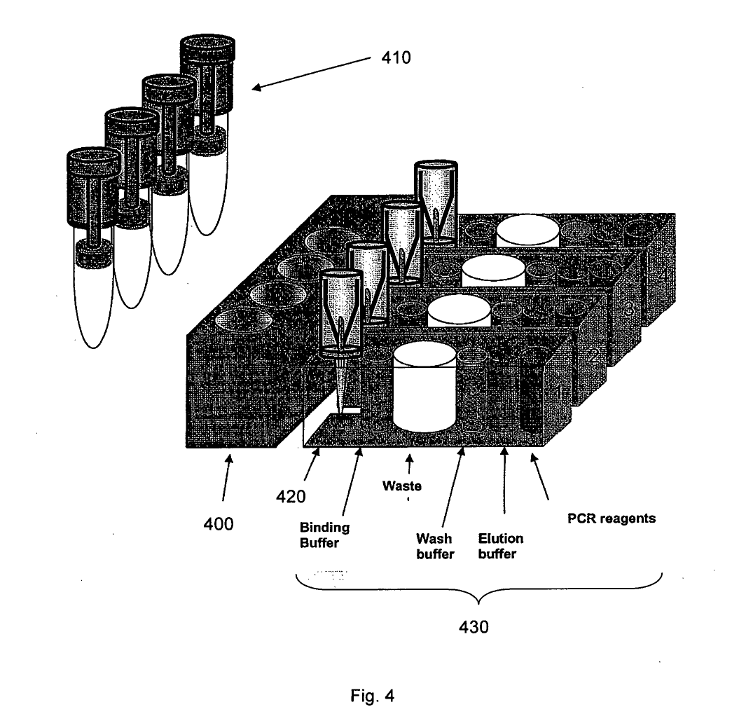

The first, second, and third racks 210, 240, and 260 may be disposed separately, combined in groups (e.g. the first rack by itself and the second and third racks together), or configured together in an array in accordance with the system design. As an example, to facilitate the multiple simultaneous processing of the disposable tube devices 100, FIG. 4 shows a sonicator horn 400, an array of sonication tubes 410, with the appropriate number of wells for accepting the sonication tubes 410, an array of filter units 420, and an array of reagent cartridges 430. Note that the system 200 is not limited to these array configurations. For example, the array of reagent cartridges (binding buffer, waste, wash buffer, and PCR reagents) 430 may be circularly configured and the robotic arm 220 may be appropriately configured to accommodate the circular array of reagent cartridges.

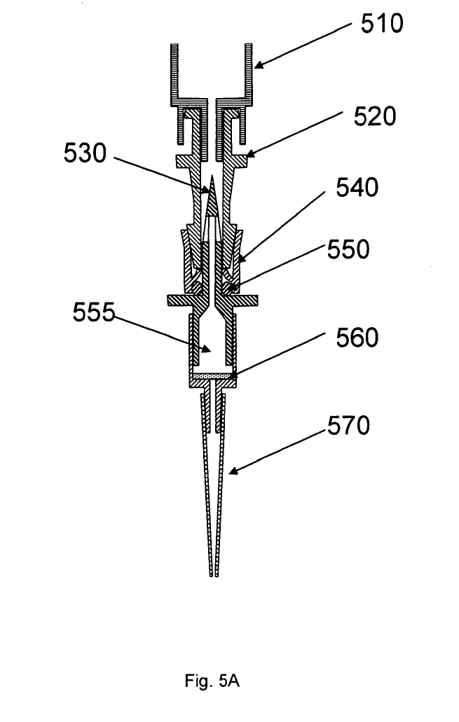

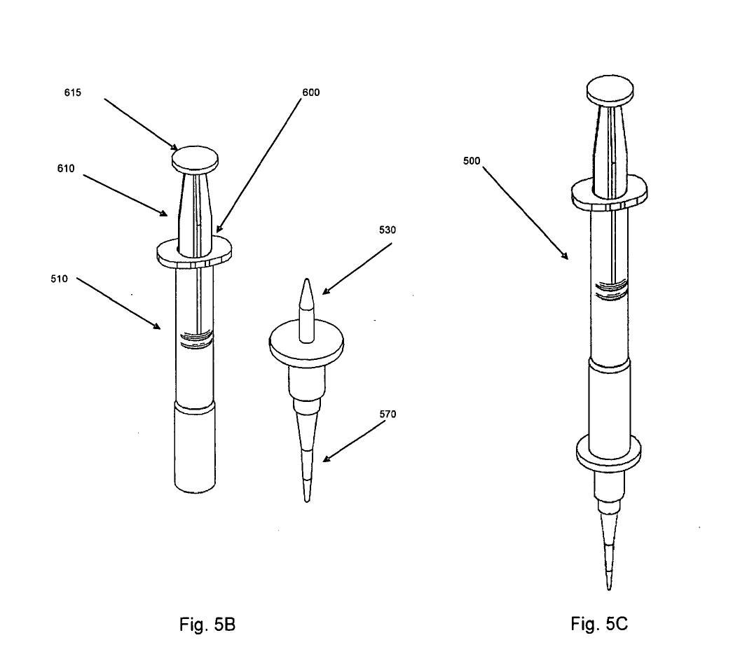

FIG. 5A shows another embodiment of a disposable tube device. A cross section of a disposable tube device 500 is shown having a syringe 510, a barb adapter 520, a piercing member 530, a bottom portion 540, an O-ring 550, a filter 560, and a tip 570. The syringe 510 has a plunger above (not shown) and is connected to the barb adapter 520, which is in turn connected to the bottom portion 540 which has a pierceable membrane at its bottom or near it. The pierceable membrane can be a septum. The sample solution is contained within the top half of the disposable tube device 500 described above. The piercing member 530 sits above a chamber 555. Disposed below the chamber 555 is the filter 560, which is an absorbing member for binding substances. A plain glass fiber membrane can be used as filter 560. The tip 570, which is a tapering tube, is attached to the chamber 555 with the filter 560 interposed there between. This configuration is the bottom half of the disposable tube device 500. The O-ring 550 ensures an air tight connection when the top half and the bottom half are connected. In the above sample solution, reagents can be added to absorb the target substances. The exemplary reagents include chaotropic agent and alcohols as disclosed in JP2006-87394 and JP1994-205676, which are incorporated herein by reference.

FIGS. 5B and 5C show the top half (a sonication tube) and the bottom half (a filter unit) separately (FIG. 5B) and together (FIG. 5C). As seen in these figures, a circular flange 600 is provided in the syringe 510 of the disposable tube device 500. A plunger 610 as shown has a cap 615. Although the example shows the circular flange 600, the configuration is not limited to this. For example, the protrusion may be configured into two separate flanges protruding in opposite directions. In an exemplary system described below, the circular flange 600 can be used by a robotic arm to hold the disposable tube device 500.

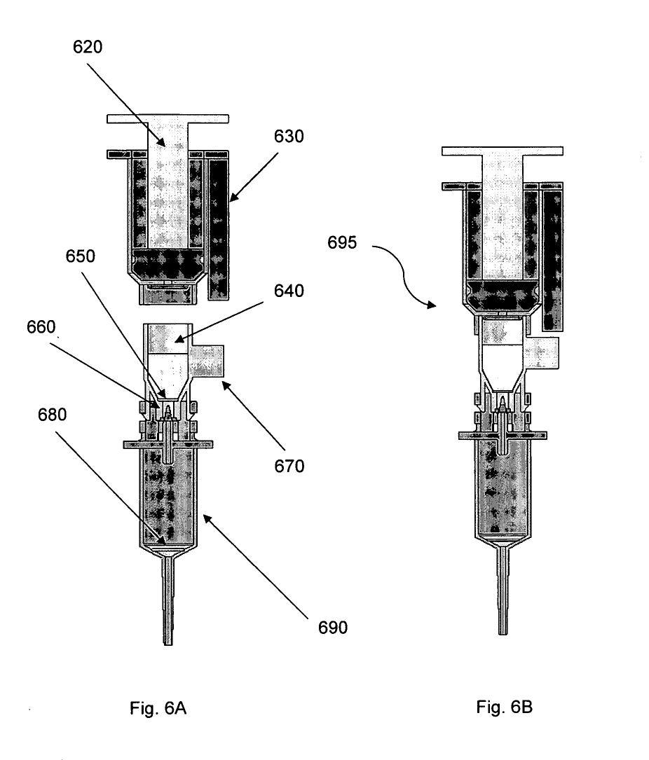

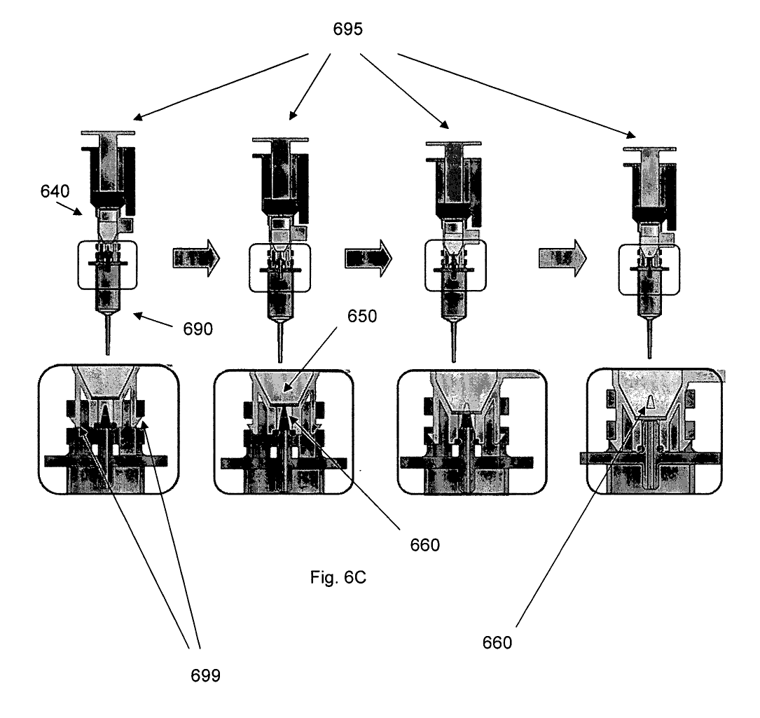

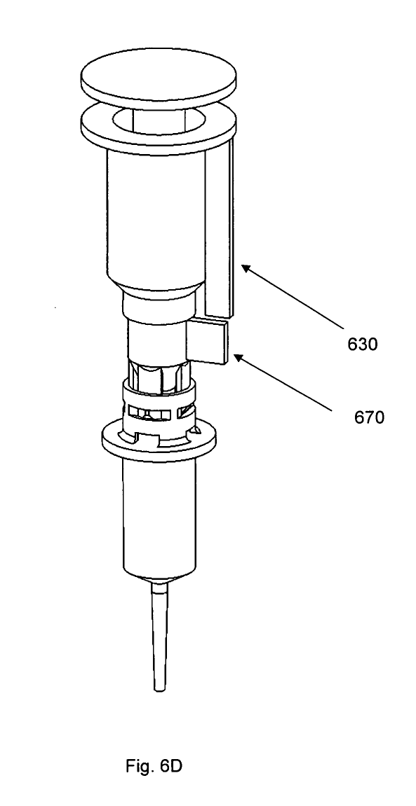

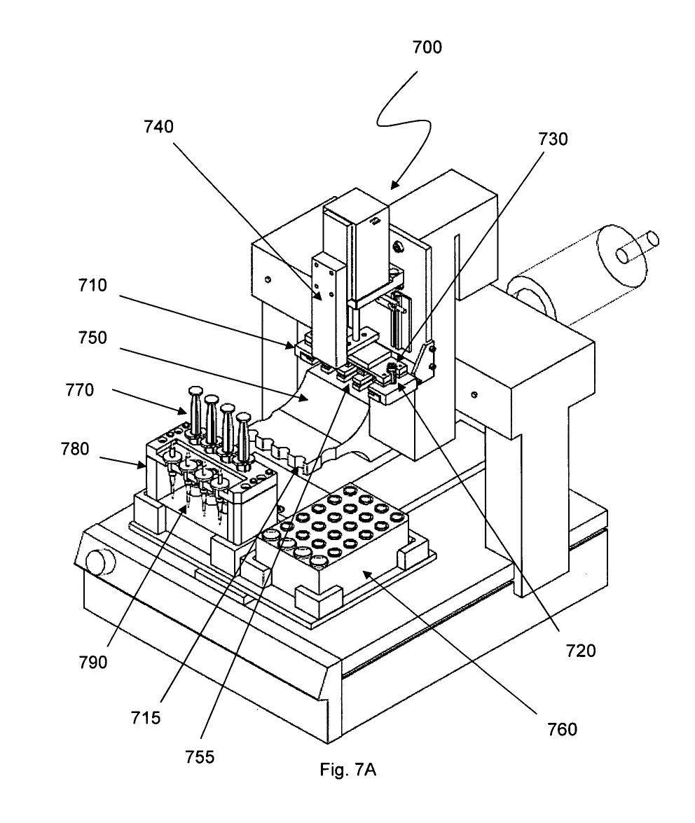

FIG. 6A-6C show another embodiment of a sonication tube. In the cross-sectional view of FIG. 6A, a plunger section 620, although not limited to this configuration, is made to screw into a sample section 640 containing a sample solution. The sample section 640 may hold about 1 ml by volume. The plunger section 620, which may hold about 5 ml by volume, has a first fin 630 for alignment. A bottom portion 650 of the sample section 640 is made to be pierced by a piercing member 660. The sample section 640 has a second fin 670 that is to be aligned with the first fin 630. When the fins 630 and 670 are aligned, an air-tight sealed is achieved. A tube section 690, which may hold about 3 ml by volume, provides a base support to the piercing member 660. An absorption member 680 is located at the bottom of the tube section 690. FIG. 6B shows a state of the sonication tube 695 where the plunger section 620 has been attached to the sample section 640 but without piercing the bottom portion 650. The sample section 640 may be sonicated before attaching the plunger section 620. The total length of the sonication tube 695 may be about 125 mm and the top base of the plunger section 620 may be about 30 mm in diameter.

FIG. 6C shows a cross-sectional view of how the piercing member 660 pierces the bottom portion 650 by pushing the sample section 640 via the plunger section 620 into the tube section 690. A locking mechanism 699 with hooks assures that once the sample section 640 is pushed into the tube section 690 and locked in, the piercing member 660 has pierced the bottom portion 650 of the sample section 640. FIG. 6D, shows a perspective view of the sonication tube device where the first fin 630 and the second fin 670 have been aligned. Except for the attachment of the plunger section 620 to the sample section 640, the process of the preparing the test sample via a robotic arm is the same as described previously. That is, sonication can be applied after the plunger section 620 and the sample section 640 have been combined but before the bottom portion 650 has been pierced.

An embodiment of a robotic arm assembly 700 is illustrated in FIGS. 7A-7E (see FIGS. 5A-5C, and 6A-6D for the disposable tube device), which includes two sandwiching plates 710 having U-shape cut outs 755—a gripping mechanism to hold the disposable tube devices 500, springs 720 to hold the two sandwiching plates 710 together and to clamp down on the flanges 600 of the disposable tube devices 500 when the devices are disposed in the U-shape cut outs 755, and a plunger holder 730 attached to a plunger actuator 740. The robotic arm assembly 700 is designed with actuators to move right or left, up or down, and forward or backward in order to freely transport the disposable tube devices. The plunger actuator 740 moves the plunger holder 730 vertically relative to the sandwiching plates 710 so that the movements of the plunger holder by the actuator allow for the plunger 610 to slide up and down relative to the disposable tube device 500. The gripping mechanism described here is one embodiment and is not limited to this. The figure also shows a buffer/reagent rack 760 where the robotic arm assembly 700 can bring the disposable tube devices 500 for picking up the solution. The gripping mechanism may be configured in coordination with how the disposable tube device is arranged. For example, the disposable tube device may have a latching mechanism to latch onto the gripping mechanism so that the disposable tube device may be securely attached during its processing.

FIG. 7A further shows a sonicator horn 750 which can retract or extend to engage via grooves 715 with the sonication tubes 770 behind a rack 780, which securely holds the tubes. Once the sonication is done, the sonication horn 750 can retract and the robotic arm assembly 700 can pick up the sonication tubes 770 as shown in FIG. 7B. After the sonication tubes 770 are joined with the filter units 790 to form the disposable tube devices 795, as shown in FIG. 7C the robotic arm assembly 700 repositions the disposable tube devices over the buffer/reagent rack.

FIG. 7D shows a more detail perspective view of the robotic arm assembly 700. The plunger holder 730 is designed to hook the cap 615 of the plunger 610 while the circular flange 600 is held by the sandwiching plates 710. As shown in FIG. 7E, when the sandwiching plates 710 go forward to accept the disposable tube devices 500 into the U-shape cut outs 755, the top plate of the plates 710 at its peripheries goes over ball bumps 790 fixed to a base to push the top plate apart from the bottom plate. The circular flange 600 are slid between the plates 710 while the disposable tube device 500 is scooped into the U-shape cut out 755 and are clamped down by the springs 720 after the plates 710 retract from the ball bumps. A limit sensor 745 is also provided to limit the movement of the plunger holder 730.

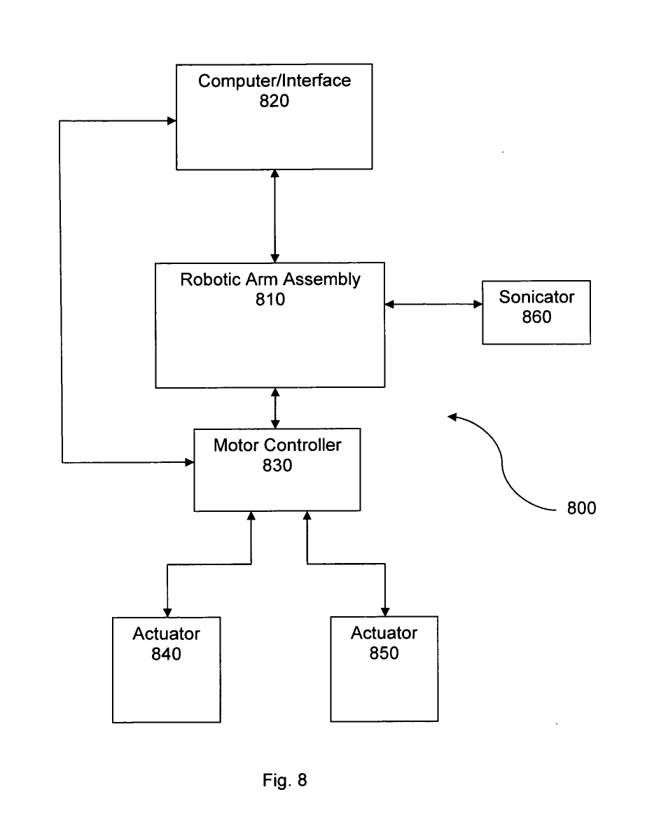

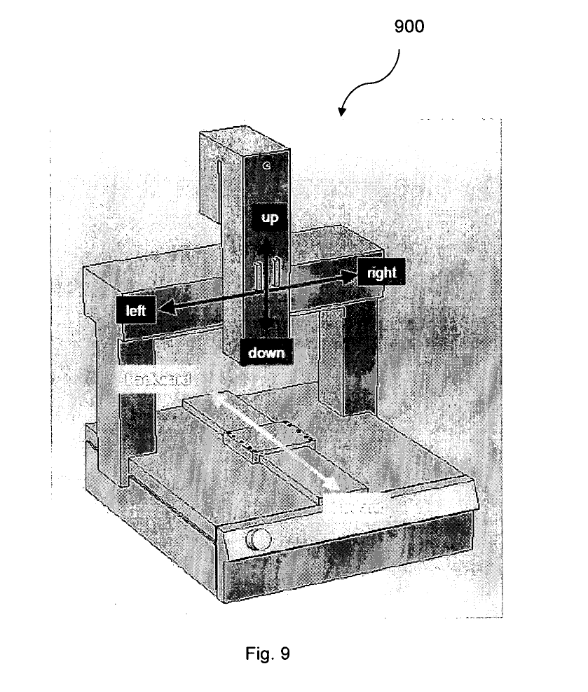

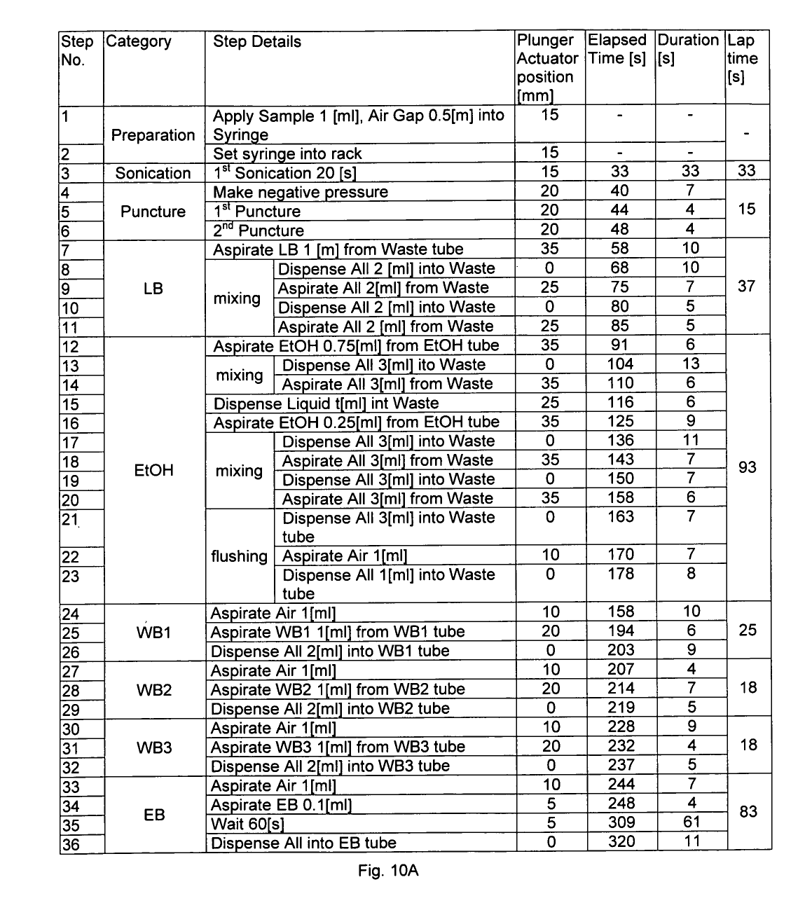

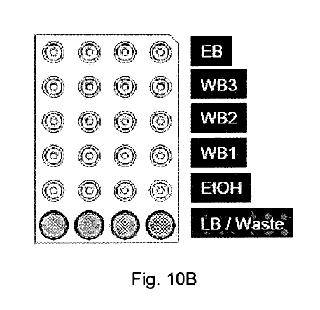

FIG. 8 shows an exemplary system 800 of the present invention for processing disposable tube devices. A robotic arm assembly 810 is controlled by a computer/interface 820 and a motor controller 830. The computer/interface 820 may be used to create internal programs for the motor controller 820 to control the robotic arm 810. Actuators 840 and 850 are motors which are under control of the motor controller 830 to drive the robotic arm 810 including the plunger (not shown) of the disposable tube device. The motor controller 830 may drive the motors to give the robotic arm the up and down, left and right, and forward and backward movements. An exemplary mechanical base 900 of the robotic arm 810 as shown in FIG. 9 illustrates how these movements are accomplished. Referring back to FIG. 8, a sonicator 860 is provided to the system 800 so that the robotic arm 810 can move the sample solutions to be sonicated. An assay protocol may be generated by the computer/interface 820 for managing the drive of the robotic arm, and an example of this is shown in FIGS. 10A and 10B. FIG. 10A shows an assay script with Step Category, Detail, Plunger position, and Duration in seconds. FIG. 10B shows the identity and layout of the reagents in a rack accessed by the robotic arm controlled according to the assay script which runs the assay protocol

In each of the disposable tube devices described above, a plunger was provided over a sonication tube. The disposable tube device is not limited to this configuration. For example, it can connect to a separate detachable mechanism that forms an air-tight seal over the device instead of a plunger. The aspiration of liquid into the device or the dispersion out of the device may be controlled by a pipetting mechanism (air control mechanism) such as a flexible tube that is hermitically placed on top of the tube and attached to a motor that can precisely control the air intake or outtake.

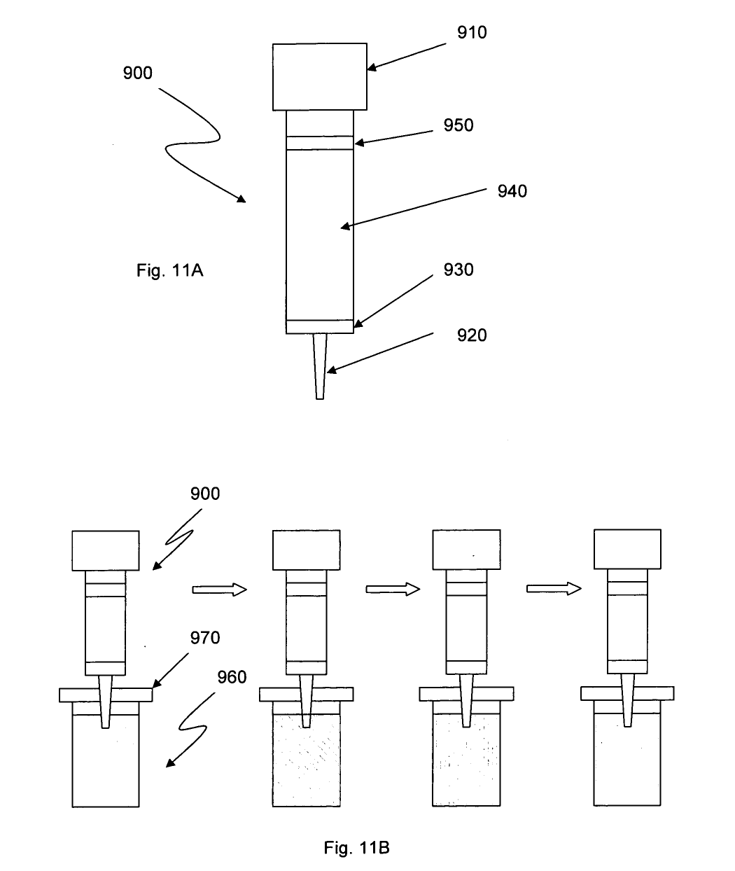

Another embodiment of a disposable tube device may be configured as shown in FIG. 11A. A disposable device 900 has a pipetting mechanism 910 sealed at the top of the device and a tapered end 920 at the opposite end for drawing in or expelling solution. The pipetting mechanism 910 may be a plunger or a pipetting actuator as described before. In between the two ends, there is a second filter 930 on top of the tapered end 920 and a chamber 940 with a capacity to hold about 3 ml of solution above this membrane. As explained earlier, the second filter can function to capture targeted substances in the sample solution. A plain glass fiber membrane can be used preferably as the second filter. There is also a first filter 950 matching the inner diameter of the device 900 and located above the chamber 940. The first filter can function as a stop gap measure to prevent overflowing by passing dry air, but not passing sample solution, buffers or reagents for contamination prevention. As shown in FIG. 11B, in this configuration, the disposable device 900 detachably engages with a sonication tube 960 with a septum 970 by piercing the septum 970 to draw in the sample solution in the sonication tube 960 after the cells or other substances have been sonicated in the tube 960. The septum 970 may be sealable components other than septum which can be pierced detachably by the a tapered end 920 to draw in the sample solution in the sonication tube 960. It is also possible to sonicate the disposable device 900 while engaged with the sonication tubes 960 instead of sonicating the tube 960 by itself. In the above sample solution, reagents can be added to help absorb the target substances by the second filter. The exemplary reagents include chaotropic agent and alcohols as disclosed in JP2006-87394 and JP1994-205676.

Next, the device 900 with the solution can be moved by a robotic arm assembly to another tube capped with a septum. This tube having a reagent appropriate for the next step in the process can be pierced through its septum by the tapered end 920 to draw in or expel the solution. The process can be repeated with other tubes with reagents as shown in FIG. 11B until the process such as purification of DNA is completed. The robotic arm assembly, which is automated to help draw in solution into or expel solution out from the device 900, can be configured to accommodate however the type the pipetting mechanisms is. For example, if the pipetting mechanism is a plunger, the robotic assembly can be like the one described in FIGS. 7A and 7B. If the pipetting mechanism is a pipetting actuator, the actuator can be computer controlled with the robotic arm movements so that the positive or negative pressure can be applied to the device 900 at appropriate times to such up or expel appropriate solutions.

An example of an automated genomic DNA preparation is next described with respect to the system 800 explained above. Using the system 800, three separate one ml sample solutions containing respectively 2×10⁵, 2×10⁴, and 2×10³ fresh E. coli were sonicated and DNA was purified automatically from the E. coli lysates in the sample solutions with reagents from Fujifilm's QuickGene™ DNA Whole Blood kit. All three sample solutions were simultaneously but separately processed as described below.

Three sets of reagent cartridges were prepared with each set respectively filled with lysis buffer, ethanol, wash buffer, and Elution buffer. The cartridges were then placed in the corresponding position in the reagent block 760 provided in the robotic arm assembly 810 as shown in FIG. 7A. Filter units (piercer/tips) of the deposable tube devices were positioned on their corresponding rack 780 provided in the robotic arm assembly 810. Samples of 2×10⁵, 2×10⁴, and 2×10³ of fresh E. coli culture were separately diluted in each lysis buffer (LB) medium. From each diluted bacterial culture sample solution, about 1 ml was drawn into a 3-ml sonfication tube. The position of a plunger in each sonication tube was adjusted such that there was a 0.5 cm air gap above the liquid. The sonication tubes were placed on the sample rack 780 of the assembly 800, and the automated sonication sequence was activated to sonicate for 20 seconds, with a power output of approximately 47.5 W with 950 J. After the sonication, the released genomic DNA was purified by the system 800 in a manner described earlier in this specification. The purification method involved an initial binding of DNA to a membrane in a filter unit followed by 3 washing steps. The purified DNA was eluted from the membrane using 100 μl of elution buffer.

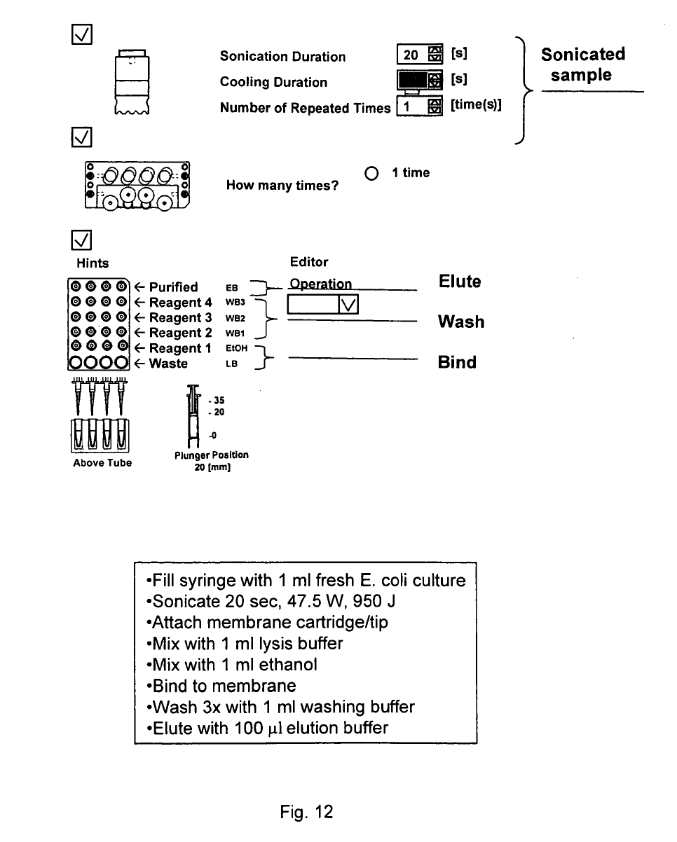

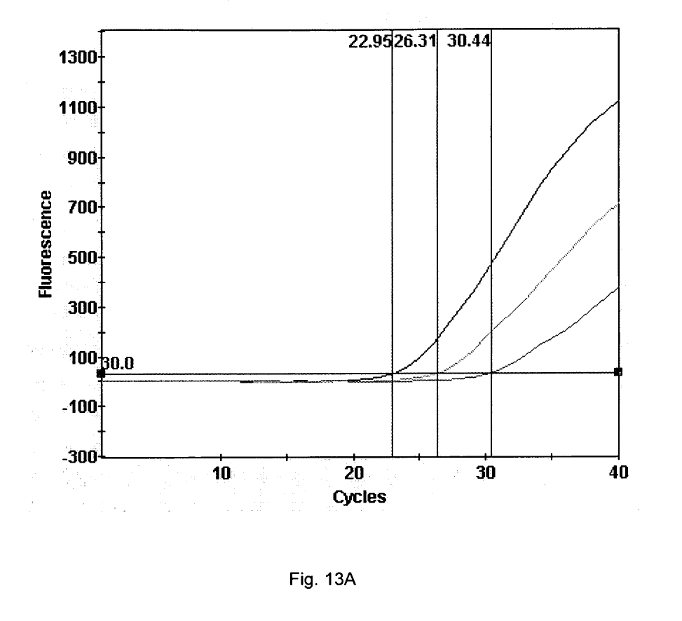

The sonication/purification method was carried out through the computer interface 820 where one example is shown in FIG. 12. The FIG. 12 interface shows three broad sections of control: sonication, puncturing; and load handling. In the sonication section, the sonication duration, cooling duration, and repeating cycles for the sonication sample solution were controlled by entering appropriate numbers into the interface. In the puncturing section, the user was given an option to select puncturing either zero or one time. The puncturing process combines the deposable tube device into a single connected unit with its septum pierced. In the load handling section, sequences of hits with the reagent/buffer solutions were controlled. As noted, the plunger position was also set by the interface. The interface is not limited to the example shown here and other various parameters can and were controlled by the interface.

One μl of the eluate was then used for performing quantitative PCR on a thermocycler (Smart Cycler™). The target was E. coli 16S ribosomal RNA gene sequence, 381-bp. The forward PCR primer was AACTGGAGGAAGGTGGGGAT and the reverse PCR primer was AGGAGGTGATCCAACCGCA. For the PCR, the following solutions (a total of about 25 μl) and conditions were used: 1×Taq polymerase buffer (10 mM Tris pH 8.8, 50 mM KCl, 0.08% Nonidat P40); 0.4 mM dNTP; 3 mM MgCl₂; 500/500 nM Forward primer/Reverse primer; 100 μg/ml BSA; 1×EvaGreen; 1 μl sample; 1 U Fermentas Taq DNA polymerase; and 95° C. (10 s)-58° C. (15 s)-72° C. (10 s) 40×.

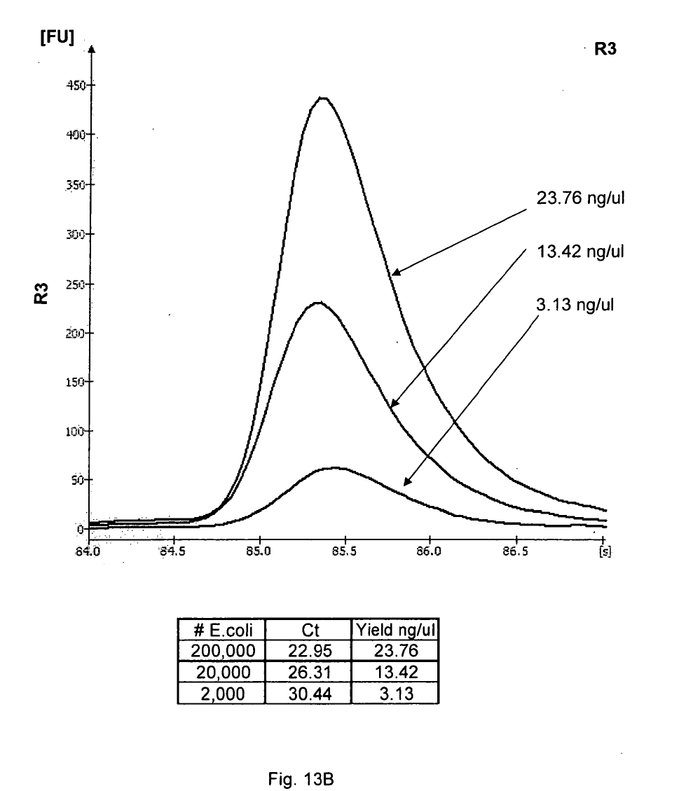

After the PCR cycles were finished, 1 μl of the reacted solution was analyzed on bioanalyzer using a DNA kit. The analysis was repeated for the other two samples. The result showed concentration dependent growth curves as shown in FIG. 13A (quantitative PCR growth curve) and 13B (bioanalyzer electropherogram) for the three samples. The yields of PCR products were determined to be 23.8, 13.4, and 3.1 ng/ul for the respective starting samples of 2×10⁵, 2×10⁴, and 2×10³ E. coli. The automated genomic DNA process worked to correlate the original concentrations of E. coli. to the final yields of the PCR products.

Other embodiments of the invention will be apparent to those skilled in the art from consideration of the specification and practice of the invention disclosed herein. It is intended that the specification and examples be considered as exemplary only.

What is claimed is:

1. A solution processing disposable device comprising:
a tube that is closed at one end and is hermetically sealable at the other end for storing a sample solution therein; and
a unit comprising:
a base member for receiving the tube and having a piercing member for piercing a wall of the tube to allow for fluid communication between the tube and the base member;
an absorbing member provided at an end of the base member opposite to where the tube is received, wherein the absorbing member allows absorption of substances in the sample solution; and
a fluid communicating member attached to the base member at the end away from where the tube is received for allowing fluid to communicate with the base member through the absorbing member.

2. A device according to claim 1, wherein the tube has an air control mechanism that hermetically seals over the tube; and the tube is a sonicaton tube configured to be received by a sonicator for sonication.

3. A device according to claim 1, wherein the fluid communicating member is a tapering tube for drawing in fluid into the base member and the tube or expelling fluid out of the base member and the tube through the absorbing member.

4. A device according to claim 2, wherein the air control mechanism is a plunger with a cap for hermetically sealing the tube, wherein the plunger adjustably draws in fluid into or expels fluid out of the base member and the tube after the piercing member of the base member has pierced the wall of the tube.

5. A device according to claim 4, wherein the tube has a spacer situated to space the plunger within the tube.

6. A device according to claim 1, wherein the piercing member is a needle or a pointed rod with a hollow center.

7. A device according to claim 1, wherein the base member is configured to receive the tube and within the base member the piercing member is positioned to pierce the tube when the tube is received to allow for fluid communication between the base member and the tube.

8. A device according to claim 1, the base member has a tubular form with the piercing member integrated inside the base member for piercing the wall of the tube.

9. A device according to claim 1, wherein the absorbing member is a membrane with an affinity for nucleic acids.

10. A device according to claim 1, wherein the device is for extracting or purifying nucleic acids.

11. An automatic sample solution treatment system comprising:
one or more tubes each with an air control mechanism to hermetically seal a sample solution inside;
a robotic arm for transporting the tubes;
a sonicator horn for accepting the tubes to sonicate the tubes;
one or more units respectively having piercing members, where each piercing member is in a space for receiving the tube, respectively having fluid communicating members, where each fluid communicating member is at an end opposite where the tube is to be received, and respectively having absorbing members for binding substances in the sample solution;
a gripping mechanism attached to the robotic arm for gripping the tubes so that the tubes can be securely pressed by the robotic arm into the respective units to cause the piercing members of the respective units to pierce the wall of the tubes and to thereby combine the tubes and the respective units to form single disposable tube devices, wherein for each disposable tube device, fluid communication between the tube and the fluid communicating member of the unit is made through the absorbing member;
vials and/or wells containing buffers and reagents for the robotic arm to position the disposable tube devices over the vials and/or wells; and
air control actuator for actuating the air control mechanism of each disposable tube device to draw in the buffer or reagents from the fluid communicating member and through the absorbing member or to expel fluid inside each device through the absorbing member and out through the fluid communicating member.

12. An automatic sample solution treatment system according to claim 11, wherein the air control mechanism is a plunger such that the tubes are configured to be hermetically sealed and accepted in the sonicator horn for sonication, and the plunger of each tube has a cap for hermetically sealing the sample within the tube to avoid aerosol contamination during sonication.

13. An automatic sample solution treatment system, according to claim 11, further comprising:
a first rack for accepting the plurality of tubes;
a second rack for accepting the plurality of units; and
a third rack for holding the vials and providing wells.

14. An automatic sample solution treatment system, according to claim 11, wherein the fluid communicating member is a tapering tube.

15. A method of treating sample solutions, comprising:
providing one or more sample solutions for assays into respective tubes;
sealing hermetically each tube with an air control mechanism;
applying the tubes to a sonicator;
transporting the tubes out of the sonicator to engage with respective units each having a space for receiving the tube and having, opposite the side where the tube is received, a fluid communicating member for drawing or expelling fluid, wherein each unit has a piercing member at the space for receiving the tube;
engaging the tubes respectively to the spaces of the units to form single disposable tube devices;
piercing the tubes respectively by the piercing members of the units to allow fluid communication between the tubes and the respective units;
positioning the disposable tube devices over to a rack with wells and/or vials of buffers and reagents;
actuating the air control mechanism of the devices relative to the devices to draw in fluid or expel fluid;
absorbing the substances in the samples to the absorbing members of the devices by actuating the air control mechanism to force the samples through the absorbing members;
eluting the absorbed substances from the absorbing members by drawing in elution buffer from the well and/or vials; and
positioning the devices over the well and/or vials with reagents and expelling the substances in the elution buffer to the wells and/or vials.

16. A method according to claim 15, wherein the air control mechanism is a plunger, and a robotic arm and a gripping mechanism are used to transport the disposable tube devices in unison and to move the plungers of the devices relative to the devices to draw in or expel fluid.

17. A method according to claim 15, wherein the fluid communicating member is a tapering tube.

18. A method of treating sample solutions, comprising:
providing one or more sample solutions for assays into respective tubes;
sealing hermetically each tube with an air control mechanism;
transporting the tubes to engage with respective units each having a space for receiving the tube and having, opposite the side where the tube is received, a fluid communicating member for drawing in or expelling fluid, wherein each unit has a piercing member at the space for receiving the tube;
engaging the tubes respectively to the spaces of the units to form single disposable tube devices;
applying the disposable tube devices to a sonicator;
piercing the tubes respectively by the piercing members of the units to allow fluid communication between the tubes and the respective units;
positioning the disposable tube devices over to a rack with wells and/or vials of buffers and reagents;
actuating the air control mechanism of the devices relative to the devices to draw in fluid or expel fluid;
absorbing the substances in the sample solutions to the absorbing members of the devices by moving the plungers to force the samples through the absorbing members;
eluting the absorbed substances from the absorbing members by drawing in elution buffer from the wells and/or vials; and
positioning the devices over the wells and/or vials with reagents and expelling the substances in the elution buffer to the wells and/or vials.

19. A method according to claim 18, wherein a robotic arm and a gripping mechanism are used to transport the disposable tube devices in unison and to move the plungers of the devices relative to the devices to draw in or expel fluid.

20. A method according to claim 18, wherein the fluid communicating member is a tapering tube or a pointed rod with a hollow center.

21. A solution processing disposable tube device comprising:
a first tube having a filter matching the inner diameter of the first tube, a chamber for containing fluid below the filter, and an absorbing member below the chamber for capturing substances;
an air control mechanism attached on top of the first tube for exerting air pressure to draw in or expel fluid;
a tapering end attached to the first tube below the absorbing member within the first tube for expelling or drawing in fluid therethrough;
a set of second tubes for containing sample solutions and reagents for the first tube to draw in fluid therefrom or expel fluid thereto; and each of the second tubes having a cover on top for sealing in fluid therein,
wherein the cover is configured to be pierced detachably by the tapered end of the first tube to draw in fluid therefrom or expel fluid thereto.

22. The device according to claim 21 wherein the air control mechanism is a plunger or a pipetting actuator with an electrical motor for applying positive or negative pressure.

23. The device according to claim 21, the first tube and the second set of tubes are so configured to be placed in an automatic sample preparation system such that a solution can be processed according to the automatic sample preparation system.

24. An automatic sample solution treatment system comprising:
one or more first tubes each with an air control mechanism to hermitically seal a sample inside; wherein each first tube respectively having a filter matching the inner diameter of the first tube, a chamber for containing fluid below the filter, an absorbing member below the chamber for capturing substances, and a piercing member below the absorbing member to allow for fluid communication between the first tube and a second tube;
a set of second tubes for containing solutions and reagents for each first tube to draw in fluid therefrom or expel fluid thereto; and each second tube having a cover on top for sealing in fluid therein, wherein the cover is configured to be pierced detachably by the piercing member of the first tube to draw in fluid therefrom or expel fluid thereto;
a robotic arm for transporting the first tubes;
a sonicator horn for accepting the second tubes to sonicate the second tubes;
a gripping mechanism attached to the robotic arm for gripping the first tubes so that by the robotic arm the first tubes are able to securely press into the second tubes to cause the piercing members of the first tubes to pierce detachably the covers of the second tubes, thereby achieving fluid communication between the first tubes and the second tubes; and
an air control actuator for actuating the air control mechanism of each first tube to draw in the buffer or reagents in through the piercing member and through the absorbing member or to expel fluid inside each first tube through the absorbing member and out through the piercing member.

25. An automatic sample solution treatment system according to claim 24, wherein the air control mechanism is a plunger such that first tube is configured to be hermetically sealed on the top by the plunger and open to communication with the set of second tubes through the absorbing member, the piercing member, and the cover.

26. An automatic sample solution treatment system according to claim 24, wherein the air control mechanism is a pipetting mechanism with the air control actuator being a motor for applying positive or negative air pressure.

27. An automatic sample solution treatment system, according to claim 24, further comprising:
a first rack for accepting one or more first tubes; and
a second rack for accepting the set of second tubes.

28. An automatic sample solution treatment system, according to claim 11, wherein the piercing member is a needle or a pointed rod with a hollow center.

29. A method of treating sample solutions, comprising:
providing one or more first tubes for containing fluid therein, wherein each first tube respectively having a filter matching the inner diameter of the first tube, a chamber for containing fluid below the filter, an absorbing member below the chamber for capturing substances, and a piercing member below the absorbing member to allow for fluid communication between the first tube and a second tube;
sealing hermetically each first tube with an air control mechanism;
providing one or more sample solutions for assays into a set of second tubes, each second tube having a cover on top for sealing the sample therein;
applying the second tubes to a sonicator;
pressing the first tubes by the piercing members into the respective second tubes to pierce detachably the covers of the second tubes to achieve fluid communication;
actuating the air control mechanism of the first tube to draw in fluid or expel fluid to absorb substances in the sonicated sample solutions to the absorbing members of the first tubes by actuating the air control mechanism to force the sonicated sample solutions through the absorbing members;
positioning the first tubes over a set of third tubes containing buffers and reagents, each third tube having a cover on top for sealing in fluid contained therein; and
eluting the absorbed substances from the absorbing members by drawing in elution buffer from the third tubes.

30. A method according to claim 29, wherein the air control mechanism is a plunger, and a robotic arm and a gripping mechanism are used to transport the first tubes in unison and to move the plungers of the devices relative to the devices to draw in or expel fluid.

31. A method according to claim 29, wherein the air control mechanism is a pipetting mechanism for applying positive or negative air pressure, and a robotic arm and a gripping mechanism are used to transport the first tubes in unison.

32. A method according to claim 29, wherein the piercing member is a tapering tube or a pointed rod with a hollow center.

33. A method according to claim 29, wherein the covers are septums.
